(19) United States
(12) Patent Application Publication
(10) Pub. No.: US 2012/0078293 A1
(43) Pub. Date: Mar. 29, 2012

(54) APPLICATORS FOR PATCHES AND ADHESIVES

(75) Inventors: Noam Hassidov, Western Galilee (IL); Havazelet Bianco-Peled, Tivon Mail (IL); Ohad Kimhi, Kiryat Yam (IL); Shlomo Lewkowicz, Kiryat Tivon (IL); Adi Azran, Kiryat Motzkin (IL); Eyal Hendel, Kfar Yehoshua (IL); Rebecca Bank, Ramat Ha'Sharon (IL); Rina Lev, Haifa (IL); Ronit Langzam-Sinai, Kiryat Tivon (IL); Tehilla Bar-Yehuda, Kiryat Bialik (IL)

(73) Assignee: TECHNION RESEARCH & DEVELOPMENT FOUNDATION LTD., Technion City, Haifa (IL)

(21) Appl. No.: 13/260,047

(22) PCT Filed: Mar. 28, 2010
(86) PCT No.: PCT/IL10/00268

§ 371 Date: Dec. 6, 2011

Related U.S. Application Data
(60) Provisional application No. 61/202,688, filed on Mar. 27, 2009.

(51) Int. Cl. A61B 17/03

(52) U.S. Cl. 606/213

(57) ABSTRACT

Apparatus is provided for use with a tubular structure (2) in a body of a patient. The apparatus includes an adhesive including first and second components, a container (27), one or more patches (3), and an applicator (1). The container (27) contains the first component of the adhesive and not the second component of the adhesive. The one or more patches (3) include the second component of the adhesive and not the first component of the adhesive. The applicator (1) is configured to removably hold the one or more patches (3), and to place the one or more patches (3) at least partially around the tubular structure (2). Other embodiments are also described.

### CROSS-REFERENCE TO RELATED APPLICATIONS

The present application claims priority from U.S. Provisional Application 61/202,688, filed Mar. 27, 2009, entitled, “Applicators for patches and adhesives,” which is incorporated herein by reference.

### FIELD OF THE APPLICATION

The present invention relates to an applicator system for applying adhesives or sealants compound to tissues and other structures in vivo.

### BACKGROUND OF THE APPLICATION

Tissue adhesives have been increasingly used to enhance traditional closure technologies such as sutures and staples, offering improved sealing capabilities and plugging of undesired leaks. Using an adhesive for tissue reattachments or repair procedures usually requires the adhesive to be applied onto a hydrated tissue surface. The method of applying the adhesive materials influences the effectiveness of the adhesive.

PCT Publication WO 06/092798 to Bianco-Peled et al. and US Patent Application Publication 2008/0167400, which are incorporated herein by reference, describe a novel composition-of-matter, method of manufacturing thereof, and applications thereof as an adhesive, in a wide variety of different fields, and in particular, in the health care fields of medicine, dentistry, and veterinary science, for use by health care providers, such as medical, dental, and veterinary, surgeons, in procedures for reattaching or repairing body parts or components thereof, such as tissue, of (human or animal) subjects, especially under wet conditions, for example, involving adhesion of wet surfaces. The composition comprises a cross-linked form of a water miscible polymer, and at least one phloroglucinol type compound selected from the group consisting of phloroglucinol, a derivative of phloroglucinol, and a polymer synthetically prepared from phloroglucinol or a derivative of phloroglucinol. An exemplary water miscible polymer is a naturally existing, or synthetically prepared, salt form of the carbohydrate alginic acid, such as sodium alginate, or alginic acid itself.

PCT Publication WO 09/060438 to Bianco-Peled et al., which is incorporated herein by reference, describes an adhering bandage, especially used for medical purposes, that comprises pre-gel of phenol-based compound and water miscible polymer that is capable of interacting with a cross linking agent. This mixture is provided with a solid support to form a bandage. An in-situ method of applying adhering bandage is also described.

PCT Publication WO 09/060439 to Bianco-Peled et al., which is incorporated herein by reference, describes a composition of matter for to be used as an adhesive that comprises pre-gel made of phenol-based compound and water miscible polymer that interacts with a cross linking agent. A method of in-situ applying the adhesive is also described.

US Patent Application Publication 2002/0026159 to Zhu et al., which is incorporated herein by reference, describes a patch applicator having an elongate body with an inner lumen and an outer lumen arranged concentrically. First and second connectors are provided and correspond to the inner and outer lumens, respectively. A patch is also provided. In operation, a source of vacuum is connected to the first connector and draws a vacuum through the inner lumen. The inner lumen is then applied to a patch to releasably hold the patch. The patch is advanced within the patient until it is placed on top of and over a wound in tissue. The source of vacuum is then transferred to the second connector and pulls a vacuum through the outer lumen to clear bodily fluid from a field around the wound. After the field is cleared, the source of vacuum is removed and replaced by a source of flowable adhesive. Adhesive is injected through the outer lumen onto the patch and tissue surrounding the patch. The patch applicator holds the patch in place, allowing the adhesive to set. A release rod is advanced through the first connector and through the inner lumen into contact with the patch. The applicator is then removed from the patch, followed by the release rod. Thus, an adhesive patch is attached to close an opening in tissue.

The following patents and patent application publication, all of which are incorporated herein by reference, may be of interest:

U.S. Pat. No. 1,881,383

U.S. Pat. No. 1,992,344

U.S. Pat. No. 2,715,903

U.S. Pat. No. 3,358,682

U.S. Pat. No. 3,542,021

U.S. Pat. No. 4,374,520

U.S. Pat. No. 4,840,187

U.S. Pat. No. 5,024,217

U.S. Pat. No. 5,259,835

U.S. Pat. No. 5,368,581

U.S. Pat. No. 6,508,430

U.S. Pat. No. 6,695,515

US Patent Application Publication 2002/0052570

US Patent Application Publication 2002/0115952

US Patent Application Publication 2004/0138601

US Patent Application Publication 2004/0238559

US Patent Application Publication 2005/0153090

US Patent Application Publication 2005/0274453

US Patent Application Publication 2007/0068837

PCT Publication WO 84/01285

British Patent Application Publication GB 2 211 740 A1

British Patent Application Publication GB 2 235 134 A1

British Patent Application Publication GB 200725087A

Japanese Patent Application Publication JP 2001/309985 A2

Japanese Patent Application Publication JP 2004/337580 A

### SUMMARY OF APPLICATIONS

Embodiments of the present invention provide methods and applicators for applying one or two components of single-component or a dual-component adhesive glue or sealant to a tissue surface, thus allowing optimal on-site curing of the adhesive. During a first stage of an adhesive application procedure, a first component of the adhesive, e.g., a viscous pre-gel, is applied to the tissue surface. During a second stage of the procedure, the methods and applicators described herein are used to apply a patch (e.g., a solid patch) or other solid support to the tissue surface. The patch comprises a second component of the adhesive, e.g., a curing agent, such that the patch and curing agent together serve as a curing/hardening patch. The curing agent, upon contact with the pre-gel, causes the pre-gel to become more solid and to adhere to the tissue surface. The patch also provides mechanical support to the tissue surface. The applicator is typically used to hold the patch against the tissue surface for about one minute. The patch is typically left on the tissue surface, and eventually biodegrades.

For some applications, the methods and applicators described herein are used for applying the components of the adhesive to a blood vessel as an adjunct to suturing. The blood vessel may comprise an artery or a vein. Alternatively, the methods and applicators are used to apply the components of the adhesive to another structure, such as, but not limited to, anatomical structures, such as the gastrointestinal tract (e.g., the colon), a bronchus, or the brain (such as the dura mater), or a synthetic structure within a body of a patient, such as a synthetic graft (e.g., a tubular synthetic graft). Typically, but not necessarily, the structure is tubular. For some applications, the components of the adhesive are applied to an intersection between two tubular structures, such as two tubular body parts, e.g., at a site of an anastomosis between two blood vessels. The intersection may, for example, be an end-to-end intersection or an end-to-side intersection.

The applicators and techniques described herein are typically used during a procedure for applying the adhesive and patch to a tubular structure, e.g., a blood vessel, such as an artery, vein, and/or a synthetic graft. During a first stage of the procedure, the surgeon applies a first component of the adhesive to the blood vessel, typically manually, and/or using a syringe. During a second stage of the procedure following the first stage, one of the applicators described herein is used to place the patch at least partially around the blood vessel, such as entirely around the blood vessel.

For some applications, the first component of the adhesive comprises a viscous pre-gel, while for other applications, the first component comprises a liquid, solid, spray, or aerosol. Similarly, the second component of the adhesive may comprises a gel, liquid, solid, spray, or aerosol, which may be applied to one or more surfaces of the patch and/or be absorbed into the patch.

The method and applicators of embodiments of the present invention enable quick and accurate application of the patch to the entire tissue surface. Such quick and accurate application is important because the first component of the adhesive generally cures quickly upon contact with the curing agent. For some applications, the applicator is further configured to retract tissue away from the tissue surface prior to or during application of the curing agent. Alternatively or additionally, a separate retractor is used. For some applications, the methods and applicators bring the ends of the patch together (e.g., adjacent to one another or overlapping one another) or near one another to enable convenient coupling of the ends of the patch to the tissue and/or to one another by the surgeon. Optionally, the applicators themselves are configured to couple the ends of the patch to the tissue and/or to one another. For some applications, the applicator is used not only to apply the patch but to apply and fasten the patch, such as by applying a measured pressure on the tissue.

For some applications, the patch applicator comprises a forceps applicator, which comprises two arms coupled to one another by a hinge, and shaped so as to define respective handles. The grasping ends of the arms comprise respective flexible patch supports that face one another and are shaped to gently apply pressure to a wall of a blood vessel or other tubular structure. For some applications, two or more (e.g., exactly two) patches are applied to respective ones of the patch supports. During the second stage of the surgical procedure, as described above, the patch supports are placed at least partially around the blood vessel, and the arms of the forceps applicator are gently closed such that the flexible patch supports apply the patches to the wall of the blood vessel.

For some applications, each of the patch supports comprises a stiff back support structure, to which is coupled a yielding pad, which may comprise, for example, silicone, rubber, or a spongy material. Each of the patches is removably coupled to one of the patch supports, such that the patch rests against the yielding pad. The yielding pad enables the forceps applicator to gently apply the patch to the blood vessel. For some applications, the patch supports are configured to be removably coupled to the arms of the forceps applicator. For these applications, each of the patch supports and a patch removably coupled thereto are packaged as a unit in a protective covering. After removing the protective covering prior to or during the surgical procedure, the patch support is coupled to one of the arms of the forceps applicator.

For some applications, the techniques described herein are used in combination with methods and/or adhesive compositions described in one or more of the following patent applications, all of which are assigned to the assignee of the present application and are incorporated herein by reference:
International Application PCT/IL2008/001451, filed Nov. 5, 2008, entitled, “Adhering bandage and methods of applying the same”;
International Application PCT/IL2008/001452, filed Nov. 5, 2008, entitled, “Adhesives and methods of applying the same”; and
International Application PCT/IL2006/000289, filed Mar. 2, 2006, entitled, “Novel adhesive materials, manufacturing thereof, and applications thereof,” which published as PCT Publication WO 06/092798.

There is therefore provided, in accordance with an application of the present invention, apparatus for use with a tubular structure in a body of a patient, the apparatus including:

an adhesive including first and second components;

a container, which contains the first component of the adhesive and not the second component of the adhesive;

one or more patches, which include the second component of the adhesive and not the first component of the adhesive; and

an applicator, which is configured to removably hold the one or more patches, and to place the one or more patches at least partially around the tubular structure.

For some applications, the applicator includes one or more patch supports, which are configured to removably hold the one or more patches, and the apparatus further includes one or more protective coverings, each of which packages one of the one or more patch supports and one of the one or more patches.

For some applications, the applicator includes forceps. For some applications, the applicator further includes one or more patch supports, which are configured to hold the one or more patches.

For some applications, the one or more patch supports include two or more patch supports, and the one or more patches include two or more patches, which are removably coupled to respective patch supports. For some applications, the two or more patch supports include exactly two patch supports; the two or more patches include exactly two patches, which are removably coupled to respective surfaces of respective patch supports; the forceps includes exactly two arms; and the arms and the patch supports are configured such that when the two patch supports are coupled to the two arms, respectively, the surfaces of the patch supports face each other.

For some applications, the patch supports are removably coupled to the forceps.

For some applications, each of the patch supports includes a stiff back support structure, and a yielding pad coupled to the stiff back support structure, and one of the one or more patches is removably coupled to the patch support such that the patch rests against the yielding pad.

For some applications, the apparatus further includes one or more protective coverings, each of which packages one of the one or more patch supports and one of the one or more patches.

For some applications, the applicator includes a finger applicator, which includes a finger-coupling element, and a patch support to which the one or more patches are removably coupled. For some applications, the finger-coupling element includes a finger cap, to which the patch support is coupled. The finger cap may, for example, include a flexible material. For some applications, the patch support includes a yielding pad, to which the one or more patches are removably coupled. Optionally, the patch support further includes a stiff back support structure.

For some applications, the apparatus further includes a protective covering that inhibits liquid contact with the one or more patches. Optionally, the protective covering is removably coupled to the applicator.

For any of the applications described above, the second component of the adhesive may include a curing agent. The first component of the adhesive may include a pre-gel.

For any of the applications described above, the tubular structure may be a tubular body part. Alternatively or additionally, the tubular structure may include one or more structures selected from the group consisting of: at least one blood vessel, and at least one synthetic graft.

For any of the applications described above, the applicator may be configured to place the one or more patches at least partially around the tubular structure having a diameter of between 2 and 30 mm.

There is further provided, in accordance with an application of the present invention, a method including:

applying a first component of an adhesive to a tubular structure in a body of a patient; and

placing one or more patches, which include a second component of the adhesive, at least partially around the tubular structure, using an applicator that initially holds at least a portion of the one or more patches, such that the second component of the adhesive comes in contact with the first component of the adhesive.

For some applications, the applicator includes one or more patch supports, and placing the one or more patches includes: removing a protective covering that packages one of the one or more patch supports and one of one or more patches; and placing the one or more patches while the one or more patches are removably coupled to the one or more patches supports.

For some applications, the applicator includes forceps, and placing includes placing the one or more patches using the forceps. For some applications, the applicator includes one or more patch supports, and placing includes placing the one or more patches while the one or more patches are removably coupled to the one or more patches supports.

For some applications, placing the one or more patches using the forceps includes removably coupling the patch supports to the forceps.

For some applications, providing the one or more patches and the one or more patch supports includes providing packaging respective ones of the one or more patch supports, and respective ones of the one or more patches, in one or more protective coverings.

For some applications, the applicator includes a finger applicator, which includes a finger-coupling element, and a patch support, and placing includes coupling the finger applicator to a human finger using the finger-coupling element, and placing the one or more patches while the one or more patches are removably coupled to the patch support. For some applications, the finger-coupling element includes a finger cap, to which the patch support is coupled, and coupling the finger applicator to the finger includes placing the finger cap on the finger. The finger cap may include, for example, a flexible material. For some applications, the patch support includes a yielding pad, to which the one or more patches are removably coupled. Optionally, the patch support further includes a stiff back support structure.

For some applications, the method further includes providing a protective covering that inhibits liquid contact with the one or more patches. For some applications, the protective covering is removably coupled to the applicator, and using the applicator to place the one or more patches includes removing the protective covering before placing the one or more patches at least partially around the tubular structure.

For any of the applications described above, the second component of the adhesive may include a curing agent, and placing the one or more patches may include bringing the curing agent into contact with the first component of the adhesive. The first component of the adhesive may include a pre-gel, and applying the first component may include applying the pre-gel to the tubular structure.

For any of the applications described above, the tubular structure is a tubular body part. Alternatively or additionally, the tubular structure includes one or more structures selected from the group consisting of: at least one blood vessel, and at least one synthetic graft.

For any of the applications described above, placing the one or more patches includes placing the one or more patches at least partially around the tubular structure having a diameter of between 2 and 30 mm.

For any of the applications described above, the method may further include retracting the tubular structure from underlying tissue before applying the first component of the adhesive to the tubular structure.

There is still further provided, in accordance with an application of the present invention, apparatus including:

a patch, which includes a component of an adhesive; and

a finger applicator, which includes a finger-coupling element, and a patch support to which the patch is removably coupled.

For some applications, the finger-coupling element includes a finger cap, to which the patch support is coupled. For some applications, the finger cap includes a flexible material. For some applications, the finger cap is shaped like a thimble. For some applications, the finger cap is shaped so as to define a cavity for insertion of a human finger, which cavity is shaped so as to define an opening having a diameter of between 1 cm and 2.5 cm, and which cavity has a length along a central longitudinal axis thereof of between 1 cm and 4 cm.

For some applications, the patch support includes a yielding pad, to which the patch is removably coupled. For some applications, the patch support further includes a stiff back support structure.

For some applications, the apparatus further includes a protective covering, which packages the patch removably coupled to the finger applicator.

For any of the applications described above, the component of the adhesive may include a curing agent.

For any of the applications described above, the apparatus may further include a container, which contains a second component of the adhesive, such as a pre-gel.

There is additionally provided, in accordance with an application of the present invention, a method including:

coupling a finger applicator to a human finger using a finger-coupling element of the finger applicator; and

placing a patch, which includes a component of an adhesive, at least partially around a tubular structure in a body of a patient, using the finger applicator while the patch is removably coupled to a patch support of the finger applicator

For some applications, the finger-coupling element includes a finger cap, to which the patch support is coupled, and coupling the finger applicator to the finger includes placing the finger cap on the finger. For some applications, the finger cap includes a flexible material. For some applications, the finger cap is shaped like a thimble.

For some applications, the patch support includes a yielding pad, to which the patch is removably coupled. For some applications, the patch support further includes a stiff back support structure.

For any of the applications described above, the component of the adhesive may include a curing agent, and placing the patch includes applying the curing agent to the tubular structure using the patch.

For any of the applications described above, the method may further include applying a second component of the adhesive to the tubular structure. For some applications, the second component of the adhesive includes a pre-gel, and applying the second component includes applying the pre-gel to the tubular structure.

For any of the applications described above, the tubular structure may be a tubular body part.

For any of the applications described above, the tubular structure may be one or more structures selected from the group consisting of: at least one blood vessel, and at least one synthetic graft.

There is still additionally provided, in accordance with an application of the present invention, apparatus including:

a patch, which includes an adhesive curing agent; and

a patch support, to which the patch is removably coupled.

For some applications, the patch support includes a coupling element.

For some applications, the patch support includes a stiff back support structure, and a yielding pad coupled to the stiff back support structure, and the patch is removably coupled to the patch support such that the patch rests against the yielding pad.

For some applications, the apparatus further includes a protective covering, which packages the patch removably coupled to the patch support.

For any of the applications described above, the apparatus may further include a container, which contains a second component of the adhesive, such as pre-gel.

There also provided, in accordance with an application of the present invention, a method including:

providing (a) a patch, which includes an adhesive curing agent, and (b) a patch support, to which the patch is removably coupled; and

coupling the patch support to an applicator.

The present invention will be more fully understood from the following detailed description of applications thereof, taken together with the drawings, in which:

### BRIEF DESCRIPTION OF THE DRAWINGS

FIGS. 1A-B are schematic illustrations of a forceps applicator in open and closed positions, respectively, in accordance with an application of the present invention;

FIG. 1C is a schematic illustration of another configuration of the forceps applicator of FIGS. 1A-B, in accordance with an application of the present invention;

FIG. 1D is a schematic cross-sectional illustration of a patch support of the forceps applicator of FIG. 1C, in accordance with an application of the present invention;

FIG. 1E is a schematic cross-sectional illustration of the packaging of the patch support of FIG. 1D and a patch, in accordance with an application of the present invention;

FIG. 1F is a schematic illustration of a kit, in accordance with an application of the present invention;

FIGS. 2A-C are schematic illustrations of a book-shaped applicator in a pre-application open position, a closed position, and a post-application open position, respectively, in accordance with an application of the present invention;

FIGS. 3A-D are schematic illustrations of a plaster applicator during respective stages of an application procedure, in accordance with an application of the present invention;

FIGS. 4A-D are cross-sectional schematic illustrations of a manual roller applicator during respective stages of an application procedure, and FIG. 4E is a perspective schematic illustration of the roller applicator, in accordance with an application of the present invention;

FIG. 4F is a cross-sectional schematic illustration of a semi-automatic roller applicator, in accordance with an application of the present invention;

FIGS. 5A-C are schematic illustrations of a pincers applicator during respective stages of an application procedure, in accordance with an application of the present invention;

FIGS. 6A-C are schematic illustrations of a retractor applicator during respective stages of an application procedure, in accordance with an application of the present invention;

FIGS. 7A-C are schematic illustrations of a retractor applicator during respective stages of an application procedure, FIG. 7D is a cross-sectional view of the applicator, and FIGS. 7E-H are additional schematic illustrations of the applicator, in accordance with an application of the present invention;

FIGS. 8A-B are schematic illustrations of an end-to-end anastomosis procedure, in accordance with an application of the present invention;

FIGS. 8C-F are schematic illustrations of an end-to-side anastomosis procedure, in accordance with respective applications of the present invention;

FIGS. 9A-B are schematic illustrations of a retractor jig, in accordance with an application of the present invention;

FIGS. 10A-B are schematic illustrations of a variable curvature applicator, and FIG. 10C is a cross-sectional view of the applicator, in accordance with an application of the present invention;

FIGS. 11A-B are schematic illustrations of a hinged applicator, in accordance with an application of the present invention;

FIGS. 12A-B are schematic perspective illustrations, and FIG. 12C is a schematic cross-sectional illustration, of a retractor book-shaped applicator, in accordance with an application of the present invention; and

FIGS. 13A-C are schematic perspective illustrations of a finger applicator, in accordance with an application of the present invention.

### DETAILED DESCRIPTION OF APPLICATIONS

In some embodiments of the present invention, during a first stage of an application procedure, a first substance is applied to a structure in a body of a patient, such a tubular structure, e.g., a blood vessel, such as manually, and/or using a syringe. Typically, the first stage further includes, before applying the first substance, retracting the tubular structure from underlying tissue, either using one of the applicators described herein, or a conventional surgical tool. During a second stage of the procedure following the first stage, an applicator is used to apply a patch to the blood vessel. For some applications, the surgeon places the applicator between the blood vessel and the underlying tissue. For some applications, the patch comprises a second substance, which, upon application of the patch to the blood vessel, is brought into contact with the first substance and the blood vessel.

For some applications, the first substance comprises a first component of an adhesive (i.e., as a glue or sealant). For some applications, the first component comprises a viscous pre-gel, while for other applications, the first component comprises a liquid, solid, spray, or aerosol. Alternatively, the first substance does not comprise an adhesive, and may comprise a viscous pre-gel, liquid, solid, spray, or aerosol.

For some applications, the second substance comprises a second component of the adhesive, typically a curing agent, which may comprise a gel, liquid, solid, spray, or aerosol, which may be applied to one or more surfaces of the patch and/or be absorbed into the patch. Upon contact with the first component of the adhesive, the curing agent causes the glue to become more solid, such as a flexible solid gel, and to adhere to the blood vessel. The patch also provides mechanical support to the blood vessel. The patch is typically left on the blood vessel, and eventually biodegrades.

For some applications, such as described in above-mentioned International Application PCT/IL2008/001451, the first component of the adhesive comprises a pre-gel that comprises at least one phenol-based compound, and at least one water miscible polymer. The second component of the adhesive comprises at least one cross linking agent capable of interacting with the at least one water miscible polymer. For some applications, the patch comprises a thin film such as plastic, knitted mesh of fabric made from synthetic or natural polymer, or gauze prepared form oxidized cellulose. For some applications, the pre-gel further comprises non-soluble suspended solids, which may take the form of particles such as fibers. For some applications, the at least one phenol-based compound takes a form selected from monomer, non-cross linked polymer, or cross linked polymer.

All references to blood vessels herein should be understand by way of example and not limitation. The techniques described herein for applying an adhesive to a blood vessel may alternatively or additionally be used to apply the adhesive to another structure, such as, but not limited to, an anatomical structure, such as the gastrointestinal tract (e.g., the colon), a bronchus; or the brain (such as the dura mater), or synthetic structures within a body of a patient, such as synthetic grafts (e.g., tubular synthetic grafts). Typically, but not necessarily, the structure is tubular. For some applications, the components of the adhesive are applied to an intersection between two tubular structures, such as two tubular body parts, e.g., at a site of an anastomosis between two blood vessels. The intersection may, for example, be an end-to-end intersection or an end-to-side intersection. In the present application, including in the claims, application of the components of the adhesive and one or more patches to a blood vessel is to be understood as optionally including application of the components of the adhesive and one or more patches to a site of an anastomosis between two blood vessels.

For some applications, the applicators described herein are used both to apply the first component of the adhesive during the first stage of the application procedure, and the second component (e.g., the curing agent) during the second stage of the procedure. Alternatively, the methods and applicators are used to apply the first component of the adhesive, and the second component (e.g., the curing agent) is applied using other techniques, such as manually, and/or using a syringe. Further alternatively, for some applications, a curing agent is first applied, either using the methods and applicators described herein, or using conventional techniques, and another component of the adhesive is subsequently applied, either using the methods and applicators described herein, or using conventional techniques.

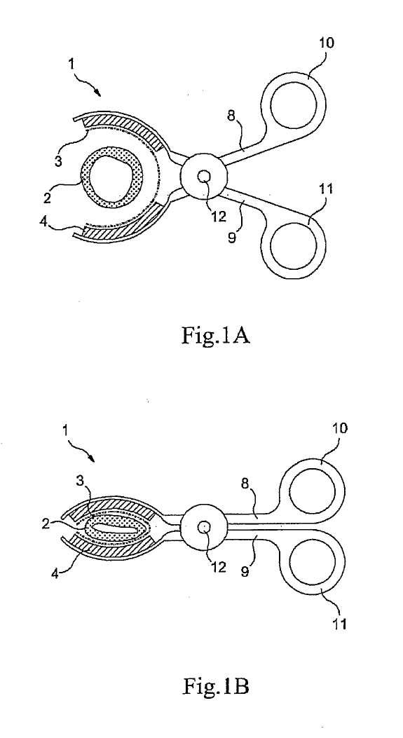

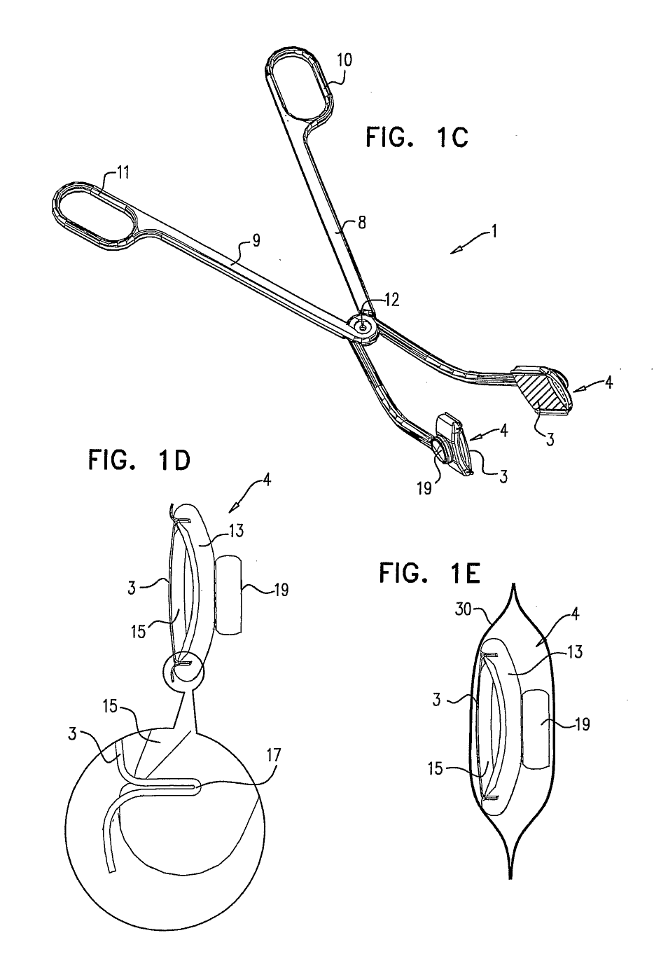

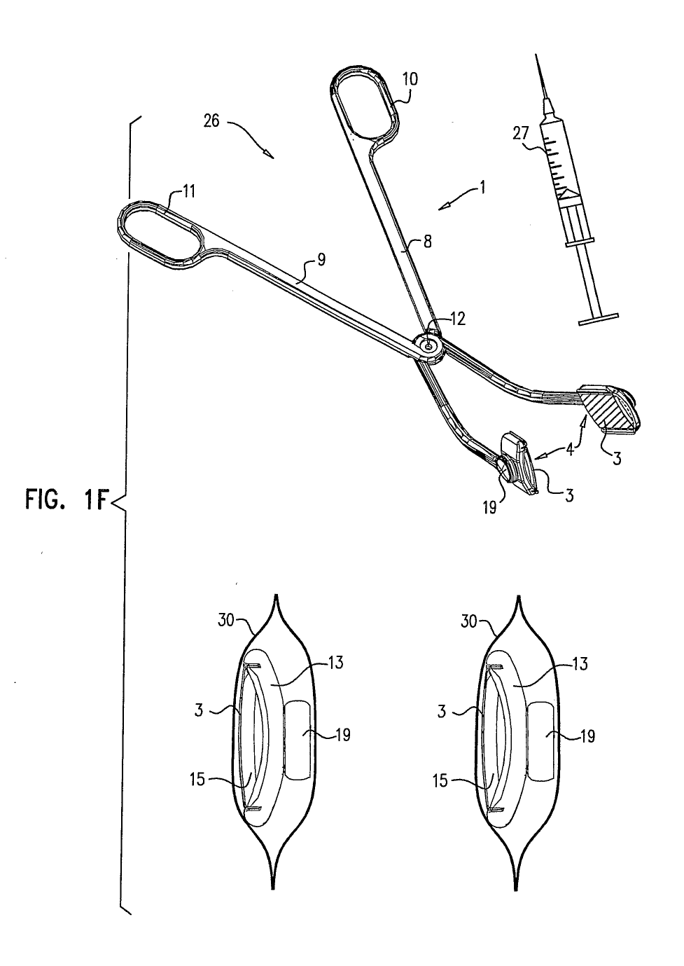

FIGS. 1A-B are schematic illustrations of a forceps applicator 1 in open and closed positions, respectively, in accordance with an application of the present invention. Forceps applicator 1 comprises two arms 8 and 9 coupled to one another by a hinge 12, and shaped so as to define respective handles 10 and 11. The grasping ends of the arms comprise respective flexible patch supports 4 that face one another and are shaped to gently apply pressure to a wall of a blood vessel 2. For example, the patch supports may be slightly curved, as shown in FIGS. 1A-B, or they may be generally flat. A single patch 3 is applied to surfaces of flexible patch supports 4 that face each another, such that the patch wraps around from one face to the other in the direction of the hinge. Alternatively, patch 3 comprises two or more (e.g., exactly two) patches, which are applied to respective ones of the patch supports, which typically face each other. Patch or patches 3 typically comprise the second substance, as described hereinabove, such as the second component of the adhesive, as described hereinabove. For some applications, forceps applicator 1 comprises a metal, such as stainless steel.

During the second stage of the application procedure, as described above, the patch supports are placed at least partially around the blood vessel, and the arms of the forceps applicator are gently closed such that the flexible patch supports apply the patch or patches to the wall of the blood vessel. For applications in which exactly one patch is applied, the flexible patch supports typically apply the patch such that ends of the patch come near or into contact with one another (either adjacent to one another or overlapping one another), as shown in FIG. 1B. Typically, the surface of each of patch supports 4 that supports patch 3 has an area of at least 1 cm2, no more than 16 cm2, and/or between 1 and 16 cm2.

FIG. 1C is a schematic illustration of another configuration of forceps applicator 1, in accordance with an application of the present invention. In this configuration, two patches 3 are provided, each of which is applied to one of patch supports 4. The patch supports typically apply the patches such that the edges of one of the patches come near or into contact with the edges of the other patch around the blood vessel.

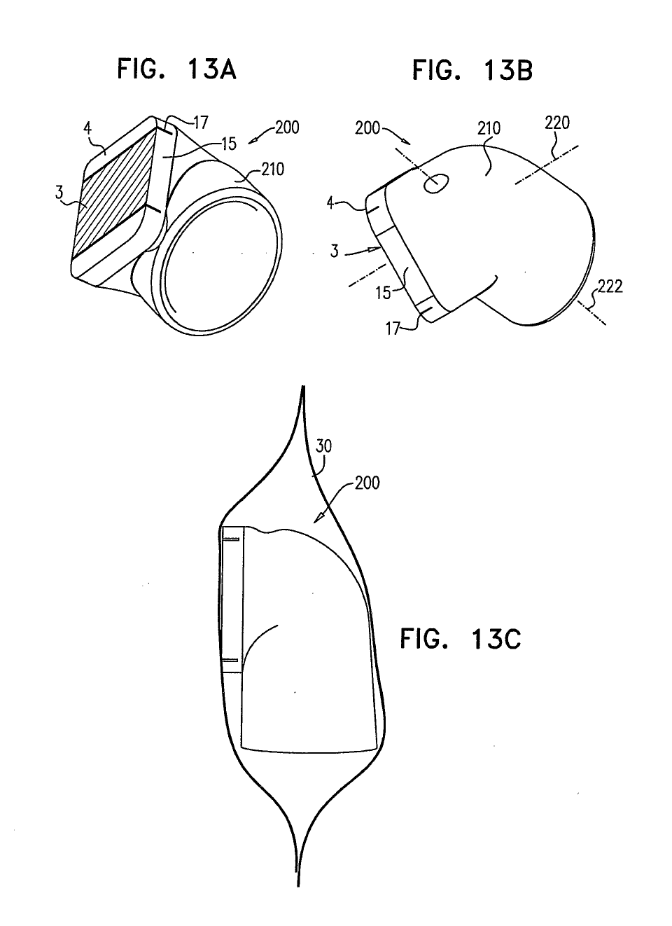

FIG. 1D is a schematic cross-sectional illustration of one of patch supports 4, in accordance with an application of the present invention. In this configuration, patch support 4 typically comprises a stiff back support structure 13, to which is coupled a yielding pad 15, which may comprise, for example, silicone, rubber, or a spongy material. Patch 3 is removably coupled to patch support 4 such that the patch rests against the yielding pad. The yielding pad enables forceps applicator 1 to gently apply the patch to the blood vessel. For some applications, back support structure 13 is shaped so as to define one or more slits 17, into which respective edges of patch 3 are inserted in order to removably couple the patch to the patch support. When the second substance of the patch is brought in contact with the first substance, the patch adheres to the blood vessel. (For other applications, such as those described hereinbelow with reference to FIGS. 13A-C, patch supports 4 do not necessarily comprise stiff back support 13.)

For some applications, patch supports 4 are configured to be removably coupled to arms 8 and 9. The patch supports are shaped so as to define respective support coupling elements, and the arms are shaped so as to define correspondingly shaped respective arm coupling elements. For example, the patch supports and arms may be configured such that the patch supports snap into the arms; the support coupling element of each patch support may be shaped as a protrusion 19, as best seen in FIG. 1D, which is inserted into a tightly couples with a corresponding opening of the arms. Alternatively, the patch supports and arms may be configured such that the patch supports slide onto the arms (configuration not shown).

FIG. 1E is a schematic cross-sectional illustration of the packaging of one of patch supports 4 and one of patches 3, in accordance with an application of the present invention. In this application, each one of the patches is packaged in a protective covering 30 together with one of the patch supports, with the patch removably coupled to the patch support. The protective covering typically completely covers the patch and patch support, in order to provide sterile packaging of the unit, and/or to reduce liquid contact with the patch. (As used in the present application, including in the claims, “liquid contact” includes liquid and moisture contact.) The surgeon removes the protective covering before coupling the patch support to the arm of forceps applicator 1, as described hereinabove.

FIG. 1F is a schematic illustration of a kit 26, in accordance with an application of the present invention. Kit 26 comprises one or more (typically two or more, e.g. exactly two) packaged patches 3 removably coupled to respective patch supports 4. Kit 26 further comprises a container 27 (e.g., a syringe or a tube), which contains a first component of an adhesive and not a second component of the adhesive; for example, the first component may comprise a viscous pre-gel. Patches 3 comprise the second component of the adhesive and not the first component of the adhesive. Optionally, kit 26 further comprises forceps applicator 1, and/or one or more of the other applicators described hereinbelow.

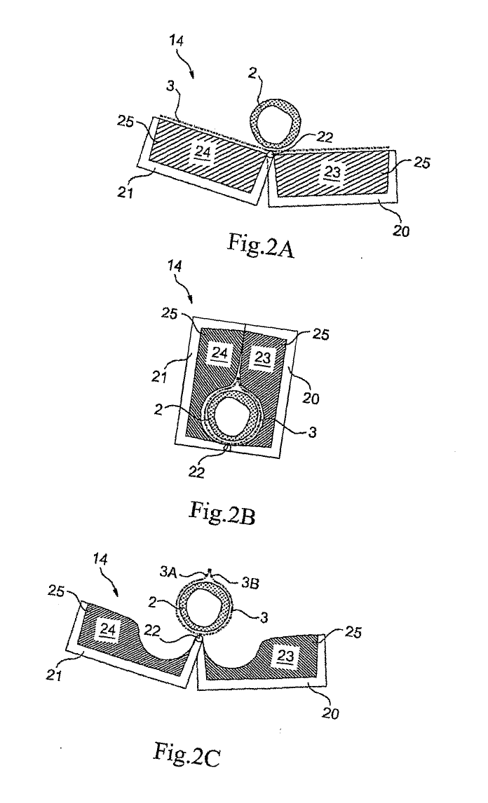

FIGS. 2A-C are schematic illustrations of a book-shaped applicator 14 in a pre-application open position, a closed position, and a post-application open position, respectively, in accordance with an application of the present invention. Book-shaped applicator 14 comprises two trays 20 and 21 coupled to one another by a hinge 22. Trays 20 and 21 are shaped to define respective cavities 23 and 24 that face one another when applicator is closed by bringing the two trays toward one another. Cavities 23 and 24 are filled with an inert flexible material 25. Patch 3 is initially positioned on an external surface of flexible material 25, as shown in FIG. 2A. During the second stage of the application procedure, the cavities 23 and 24 are brought against one another, as shown in FIG. 2B, bringing the patch around and into firm contact with blood vessel 2. Flexible material 25 is compressed to provide space for the blood vessel, as is shown in FIGS. 2B and 2C. Ends 3A and 3B of the patch are brought adjacent to one another or overlapping one another, as shown in FIG. 2C.

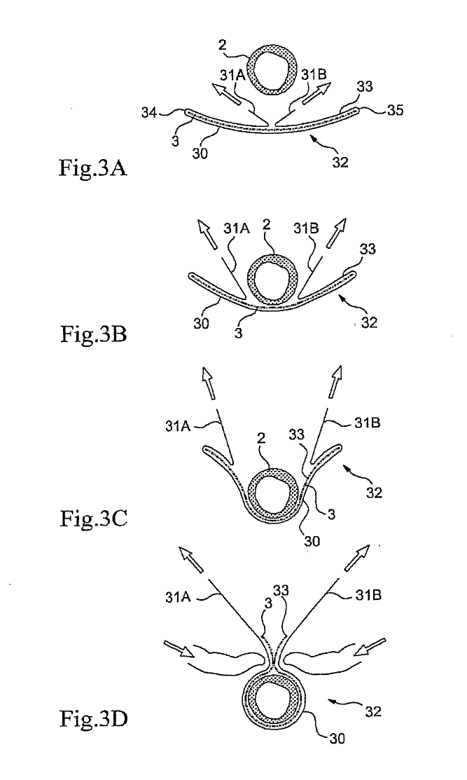

FIGS. 3A-D are schematic illustrations of a plaster applicator during respective stages of an application procedure, in accordance with an application of the present invention. Patch 3 is provided with protective covering 30 that initially surrounds the patch, covering both a lower side 32 and an upper side 33 of the patch. Protective covering 30 is initially folded over ends 34 and 35 of the patch, such that ends 31A and 31B of the protective covering initially meet in a vicinity of the center of upper side 33 of patch 3.

As shown in FIG. 3A, during the second stage of the application procedure, the surgeon first places the covered patch under blood vessel 2, between the blood vessel and underlying tissue. The surgeon uses his or her fingers and/or a surgical tool to begin pulling ends 31A and 31B of the protective covering in an upward and outward direction, which causes: (i) the protective covering to begin separating from the patch, thereby exposing upper side 33 of the patch, and (ii) the ends of the patch to move upwards toward blood vessel 2, as shown in FIG. 3B. Removal of the protective covering as the patch is applied to the blood vessel allows the protective covering to inhibit (i.e., prevent or reduce) liquid contact with the patch and the curing agent until immediately prior to application of the patch and curing agent to the surface, thereby keeping the patch dry until the patch comes in contact with the first substance on the blood vessel. For some curing agents, keeping the curing agent dry may be necessary to preserve the curing agent. For other curing agents for which this is not necessary, the applicator does not necessarily comprise the protective covering.

As the surgeon continues to pull the ends of protective covering upward, the patch begins to wrap around the blood vessel, as shown FIG. 3C, until ends 31A and 31B completely separate from the upper side of the patch and the ends of the patch come into contact and overlap one another, as shown in FIG. 3D. The surgeon then presses the ends together, such as with his or her fingers, thereby firmly wrapping the patch around the blood vessel. The surgeon removes and disposes the protective covering from lower side 32 of the patch (lower side 32 now faces outward) (step not shown). For applications in which the second component comprises a curing agent, the curing agent typically quickly cures the first component of the adhesive, such that the protective covering becomes unnecessary soon after the patch is applied.

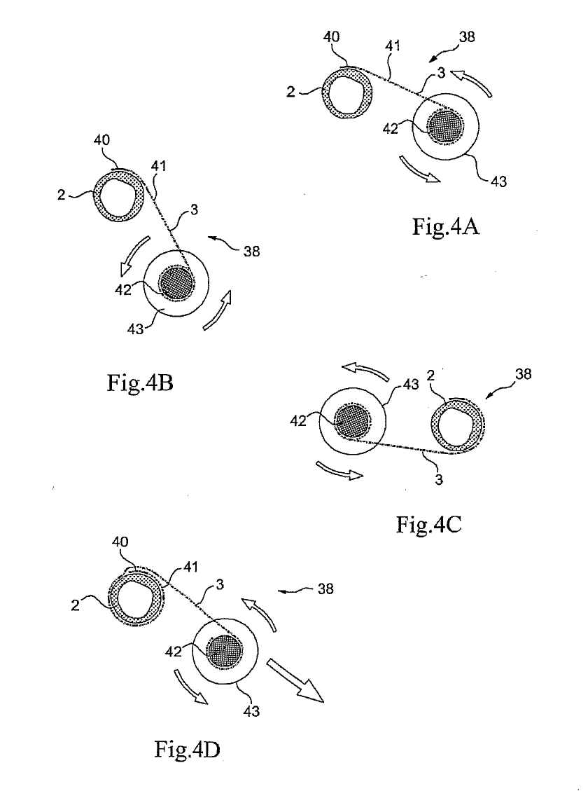

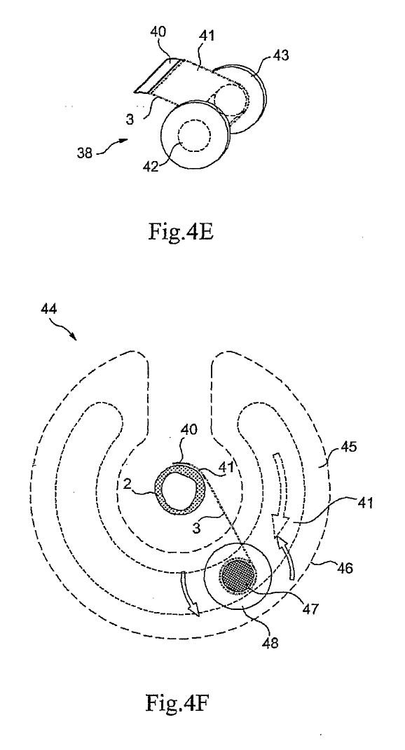

FIGS. 4A-D are cross-sectional schematic illustrations of a manual roller applicator 38 during respective stages of an application procedure, and FIG. 4E is a perspective schematic illustration of the roller applicator, in accordance with an application of the present invention. Roller applicator 38 comprises a shaft 42 and two flanges 43 (one at each end of the shaft; for clarity of illustration only a single flange is shown in the figure). For some applications, patch 3 comprises two portions: an inert portion 40 at a first end of the patch, and an active portion 41 which typically includes the entire patch except for the inert portion 40. The active portion comprises the second substance, such as the curing agent, while the inert portion does not comprise the second substance. The patch is initially rolled onto shaft 42 such that the inert portion is outermost and exposed.

As shown in FIG. 4A, during the second stage of the application procedure, the surgeon first places inert portion 40 of patch 3. The inert portion does not cure the first component of the adhesive already placed on the blood vessel during the first stage of the application procedure. Therefore, the surgeon can adjust the positioning of the first portion if necessary. In addition, for some applications, the inert portion has better mechanical properties than does the active portion. After positioning the first portion in the desired location, the surgeon rotates roller applicator 38, thereby deploying active portion 41 of patch 3 on the first component of the adhesive on the blood vessel, and wrapping the patch around the blood vessel, as shown in FIGS. 4B and 4C, until the active portion of the patch is wrapped onto inert portion 40, completely surround the blood vessel, as shown in FIG. 4D. The second component of the adhesive contained in the active portion is absorbed by inert portion 40, thereby bringing the second component in contact with the first component that was previously applied to the blood vessel.

FIG. 4F is a cross-sectional schematic illustration of a semi-automatic roller applicator 44, in accordance with an application of the present invention. Applicator 44 comprises a housing 45 to which a roller 46 is moveably coupled. Typically, the roller is positioned within the housing. Roller 46 typically comprises a shaft 47 and a flange 48. The housing is shaped so as to define a track 41 which roller 46 traverses during the second stage of the application procedure. Applicator 44 is configured to allow the surgeon to control the sliding of the roller, such as by using steppers driven by a motor or a spring, such that the deployment is slow and contorted. For some applications, applicator 44 comprises a motor that is configured to slide the roller along track 41 (configuration not shown). For some applications, patch 3 comprises inert portion 40 and active portion 41, as described hereinabove with reference to FIGS. 4A-E.

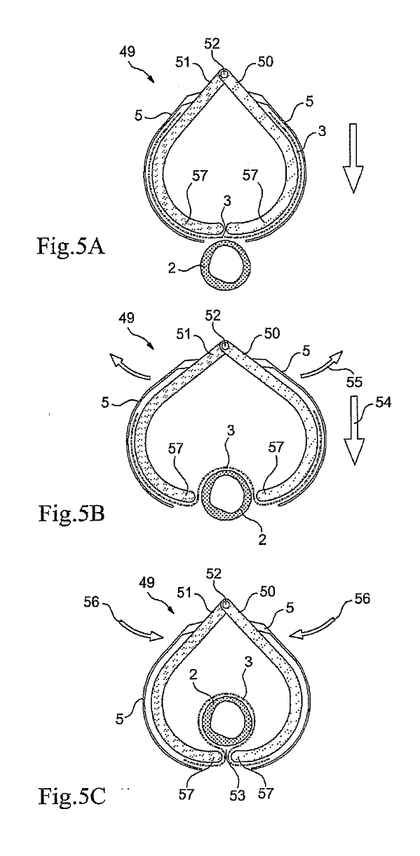

FIGS. 5A-C are schematic illustrations of a pincers applicator 49 during respective stages of an application procedure, in accordance with an application of the present invention. Pincers applicator 49 comprises two forcing arms 50 and 51 coupled to one another by a hinge 52. Arms 50 and 51 comprise respective protective cases 5 which follow the contour of the arms and define respective cavities between the cases and the arms. Patch 3 is initially placed partially within these cavities. The cases are coupled to the arms near the proximal ends of the arms (i.e., near hinge 52), and define respective opening between the cases and the arms near the proximal ends of the arms. The cases provide safe and dry storage for the patch during.

As shown in FIG. 5A, before commencement of the second stage of the application procedure, distal ends 57 of the arms are positioned near or in contact with one another. Most of patch 3 is positioned between the cases and the arms, and a small portion of the patch is exposed at the distal end of the applicator as the patch passes from one arm to the other arm.

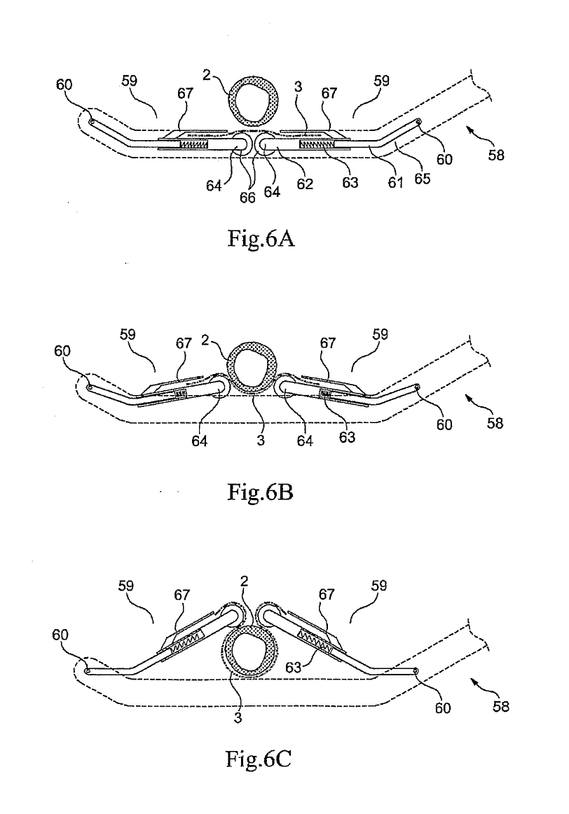

As shown in FIG. 5B, the surgeon begins the second stage of the application procedure by applying this exposed portion of patch 3 to blood vessel 2. As the surgeon gently presses down toward the blood vessel, i.e., toward the underlying tissue (as indicated by an arrow 54), while at the same time opening arms 50 and 51 (as indicated by arrows 55), the patch is deployed from the applicator and onto the surface of blood vessel 2. Distal ends 57 have a smooth shape so that they gently pass around the blood vessel. Alternatively or additionally, the distal ends comprise respective rollers, such as described hereinbelow with reference to FIGS. 6A-C.

As shown in FIG. 5C, the surgeon completes the second stage of the application procedure by continuing to gently press down toward the blood vessel, while at the same time closing arms 50 and 51 (as indicated by arrows 56). This closing motion brings opposite portion of patch 3 together at an overlap 53, thereby completely surrounding the blood vessel with the patch. For some applications, hinge 52 comprises a spring, which helps gently close or open the arms during this last phase of the procedure.

FIGS. 6A-C are schematic illustrations of a retractor applicator 58 during respective stages of an application procedure, in accordance with an application of the present invention. Applicator 58 comprises an elongated housing 65, which is shaped to serve as a retractor for separating blood vessel 2 from the underlying tissue. Applicator 58 further comprises two arm subassemblies 59 coupled by respective hinges 60 to housing 65. Each arm subassembly 59 comprises a rod 61 inserted into a slider arm 62. The rod and the slider are forced against a spring 63. Distal ends 64 of slider arms 62 have a smooth shape so that they gently pass around the blood vessel. Alternatively or additionally, the distal ends comprise respective rollers 66. Slider arms 62 comprise respective protective cases 67 which define respective cavities between the cases and the slider arms. Patch 3 is initially placed partially within these cavities. The cases provide safe and dry storage for the patch.

As shown in FIG. 6A, before commencement of the second stage of the application procedure, distal ends 64 of slider arms 62 are positioned near or in contact with one another. Most of patch 3 is positioned between the cases and the arms, and a small portion of the patch is exposed as the patch passes from one arm to the other arm. The surgeon begins the second stage of the application procedure by using housing 59 to gently retract blood vessel 2 from the underlying tissue.

As shown in FIG. 6B, the surgeon gently rotates arm assemblies 59 upward on hinges 60. The upward rotation forces the exposed portion of patch 3 against the lower side of the blood vessel, and, as distal ends 64 gently press against the blood vessel, springs 63 compress to maintain a generally constant force of the distal ends against the blood vessel. At the same time, the patch is withdrawn from protective cases 67 and is applied to the blood vessel. As the distal ends pass towards the upper side of the blood vessel, the springs decompress, holding the distal ends of the arms against the blood vessel, as shown in FIG. 6C, and bringing the ends of the patch together.

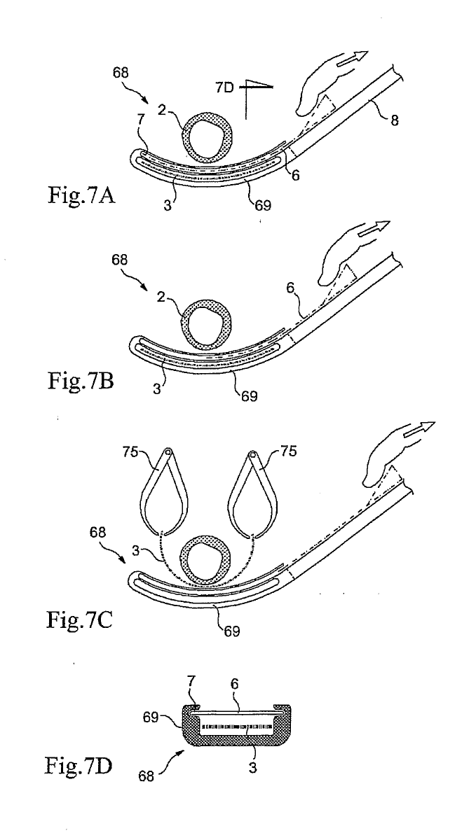

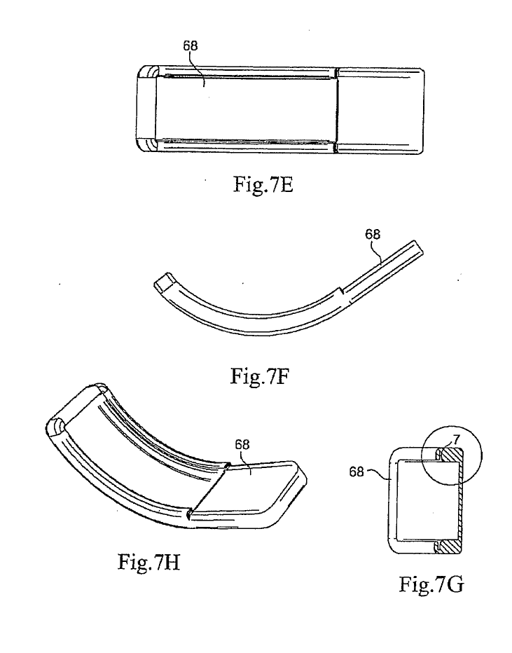

FIGS. 7A-C are schematic illustrations of a retractor applicator 68 during respective stages of an application procedure, FIG. 7D is a cross-sectional view of the applicator, and FIGS. 7E-H are additional schematic illustrations of the applicator, in accordance with an application of the present invention. Applicator 68 comprises an elongated housing 69, which, for some applications, is shaped to serve as a retractor for separating blood vessel 2 from the underlying tissue. Housing 69 is shaped so as to define two grooves 7 that are configured to hold a flexible sliding protective covering 6 over a recessed area that stores patch 3, as can best be seen in cross section in FIGS. 7D and 7G.

As shown in FIG. 7A, the surgeon begins the second stage of the application procedure by using housing 69 to gently retract blood vessel 2 from the underlying tissue. Alternatively, the surgeon first applies the applicator to the blood vessel, and subsequently applies the first component of the adhesive. Further alternatively, the surgeon uses the housing to perform the retraction before applying the first substance during the first stage of the application procedure, as described hereinabove.

As shown in FIG. 7B, once applicator 68 is in position under the blood vessel, the surgeon retracts sliding protective covering 6, exposing patch 3. Using two surgical tools 75, such as forceps, the surgeon pulls the patch upward and applies it to the blood vessel, as shown in FIG. 7C.

For some applications, before beginning the application procedures described herein with reference to FIG. 2A-C, 7A-C, 10A-B, 11A-B, or 12A-B, the surgeon cuts the patch to size in the operating room, and places the cut patch in the applicator.

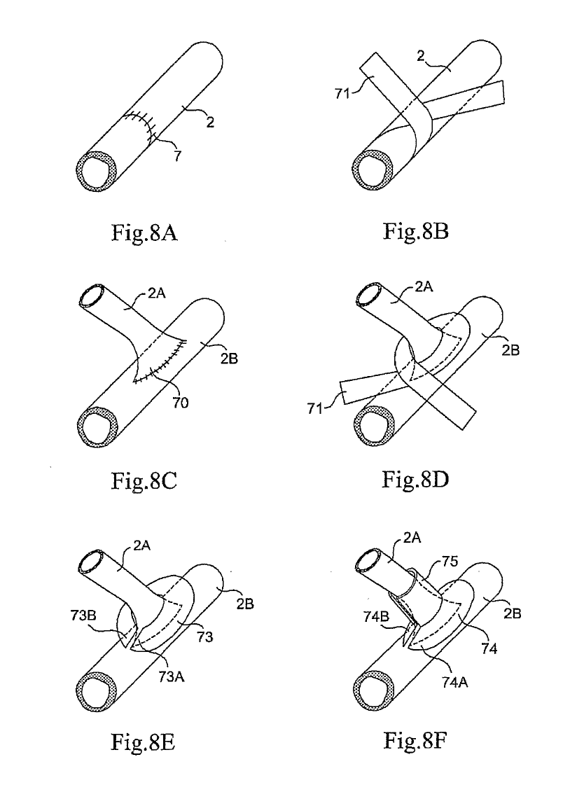

FIGS. 8A-B are schematic illustrations of an end-to-end anastomosis procedure, in accordance with an application of the present invention. During the anastomosis procedure, the ends of the blood vessel are first stitched together using conventional techniques, such as using sutures 70. During the first stage of the adhesive application procedure, the first component of the adhesive (e.g., a pre-gel) is applied to the blood vessel at the site of the anastomosis. A tie patch 71, which comprises the second component of the adhesive (e.g., a curing agent), is then manually applied by the surgeon by wrapping the patch around the blood vessel at the site of the anastomosis.

FIGS. 8C-F are schematic illustrations of an end-to-side anastomosis procedure, in accordance with respective applications of the present invention. During the anastomosis procedure, the end of a secondary blood vessel 2A is first to a primary blood vessel 2B using conventional techniques, such as using sutures 70. During the first stage of the adhesive application procedure, the first component of the adhesive (e.g., a pre-gel) is applied to the blood vessels at the site of the anastomosis.

In the application shown in FIG. 8D, a tie patch 71, which comprises the second component of the adhesive (e.g., a curing agent), is then manually applied by the surgeon by wrapping the patch around the secondary blood vessel at the site of the anastomosis.

In the application shown in FIG. 8E, a horseshoe patch 73, which comprises the second component of the adhesive (e.g., a curing agent), is then manually applied by the surgeon, by manually retracting edges 73A and 73B of the patch to create a gap, and then placing the patch around the secondary blood vessel at the site of the anastomosis.

In the application shown in FIG. 8F, a gorget-shaped patch 74, which comprises the second component of the adhesive (e.g., a curing agent), is then manually applied by the surgeon, by manually retracting edges 74A and 74B of the patch to create a gap, and then placing the patch around the secondary blood vessel at the site of the anastomosis.

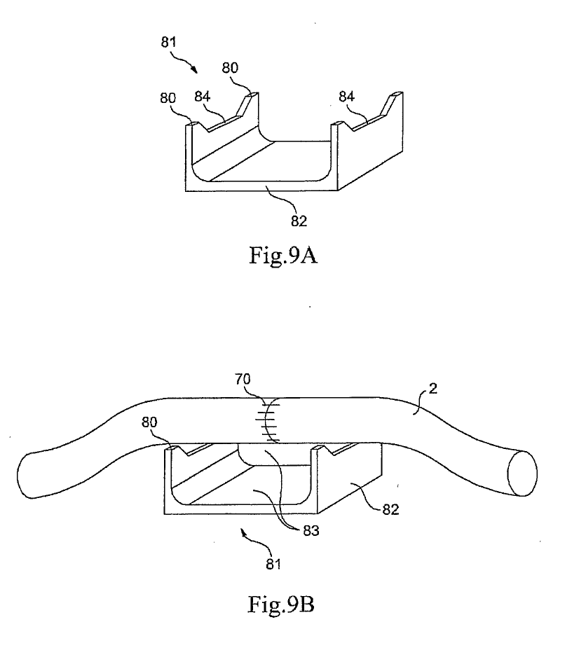

FIGS. 9A-B are schematic illustrations of a retractor jig 81, in accordance with an application of the present invention. Retractor jig 81 is configured to lift blood vessel 2 from underlying tissue and hold the blood vessel spaced from the tissue. Retractor jig 81 comprises a base 82 which supports two raised support elements 84, at respective ends of the jig. Each of the support elements typically defines two lateral border elements 80. Once retracted and placed on the jig, blood vessel 2 rests safely and firmly on support element 84, and is prevented from becoming laterally dislodged by the lateral border elements. The retractor firmly maintains a space 83 between the blood vessel and the sounding tissue.

In an application of the present invention, a method is provided that comprises providing retractor jig 81, using the jig to retract the blood vessel from underlying tissue, supporting the blood vessel with the jig, and while the blood vessel is thus supported, performing anastomosis. For some applications, performing the anastomosis comprises: (i) stitching ends of the blood vessel together; (ii) during the first stage of the application procedure, applying a first substance to the blood vessel at the site of the anastomosis, such as a first component of the adhesive (e.g., a pre-gel); and (iii) during the second stage of the application procedure, applying a second substance to the site using a patch, such as a second component of the adhesive (e.g., a curing agent). Typically, the method is performed in conjunction with application procedures and/or applicators described herein.

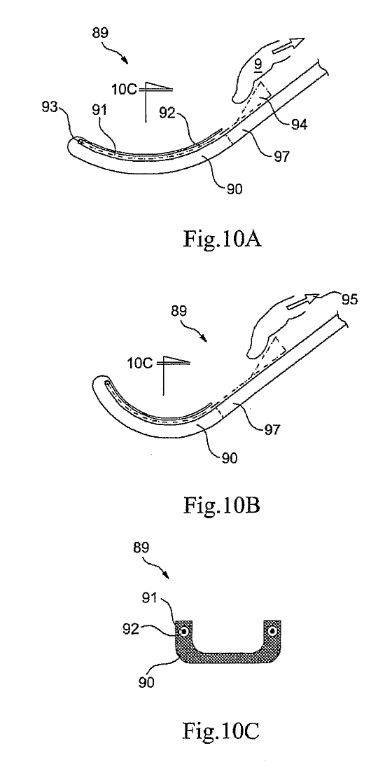

FIGS. 10A-B are schematic illustrations of a variable curvature applicator 89, and FIG. 10C is a cross-sectional view of the applicator, in accordance with an application of the present invention. Applicator 89 comprises a flexible main housing 90 that is configured to change its curvature when a force is applied thereto. The applicator comprises a flexible wire 91 that passes through an elongated cavity 92 defined by a distal portion of main housing 90. A distal end 93 of wire 91 is fixed to the main housing near a distal end of the main housing. A proximal end of the wire is fixed to a manual control 94, such as a button or tab, on a proximal handle 97 of the applicator. When a surgeon slides manual control 94 in a proximal direction (as indicated by an arrow 95), wire 91 slides through cavity 92, thereby forcing flexible main housing 90 to curve upwards.

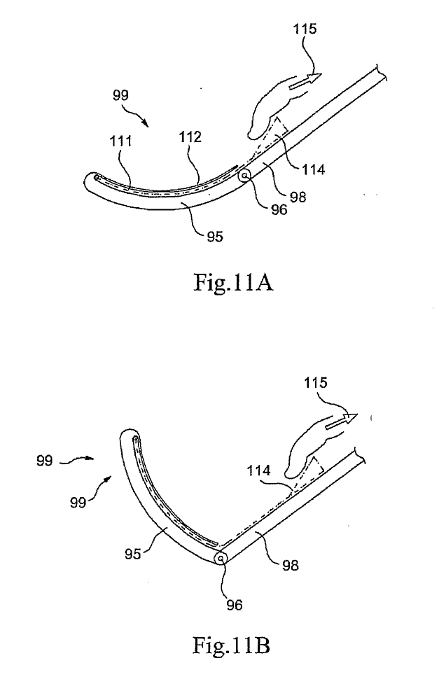

FIGS. 11A-B are schematic illustrations of a hinged applicator 99, in accordance with an application of the present invention. Applicator 99 comprises a main housing 95 coupled to a proximal handle 98 by a hinge 96. The applicator comprises a flexible wire 111 that passes through an elongated cavity 112 defined by a distal portion of main housing 95. A distal end of wire 111 is fixed to the main housing near a distal end of the main housing. A proximal end of the wire is fixed to a manual control 114, such as a button or tab, on proximal handle 98 of the applicator. When a surgeon slides manual control 114 in a proximal direction (as indicated by an arrow 115), wire 111 slides through cavity 112, thereby causing upward rotational movement of main housing 95 with respect to handle 98. This upward rotational movement may be helpful, for example, for accessing deep cavities, such as the aorta.

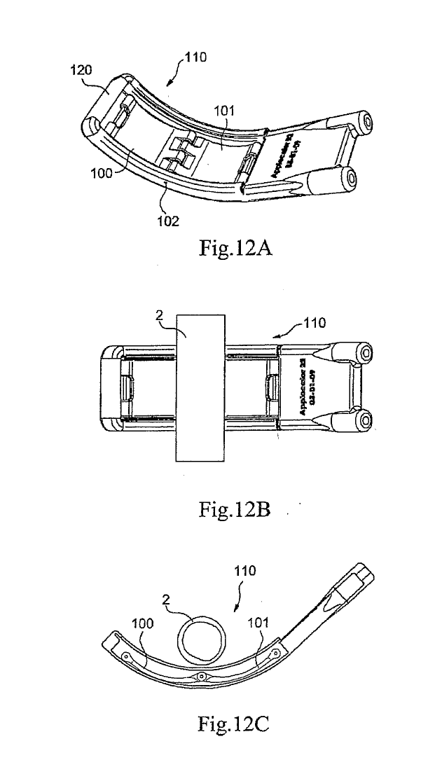

FIGS. 12A-B are schematic perspective illustrations, and FIG. 12C is a schematic cross-sectional illustration, of a retractor book-shaped applicator 110, in accordance with an application of the present invention. Applicator 110 serves both as a retractor, as described hereinabove, and a patch applicator. The applicator comprises two flexible trays 100 and 101 coupled to one another by a hinge 102. The trays and hinge are positioned within a main housing 120. The leafs may be manually operated by a surgical tool, such as pincers, surgical tweezers, or forceps, or another mechanical mechanism. After the applicator has been used to retract blood vessel 2 from underlying tissue, applicator 110 operates in a similar manner to book-shaped applicator 14, described hereinabove with reference to FIGS. 2A-C. Typically, a protective covering is provided (not shown in FIGS. 12A-B), and is removed such as described hereinabove with reference to FIGS. 7A-C.

Reference is made to FIGS. 13A-B, which are schematic perspective illustrations of a finger applicator 200, in accordance with an application of the present invention. Finger applicator 200 comprises patch support 4 and a finger-coupling element. In this application, patch support 4 typically comprises yielding pad 15, which may comprise, for example, silicone or a spongy material. For some applications, the surgeon's finger, coupled to patch support 4 by the finger-coupling element, serves to provide a stiff back support structure to yielding pad 15, and patch support 4 does not comprise a separate stiff back support structure 13. Alternatively, patch support 4 further comprises stiff back support structure 13 (configuration not shown). For some applications, the finger-coupling element comprises a flexible band that is placed around the surgeon's finger.

For some applications, the finger-coupling element comprises a finger cap 210 coupled to patch support 4. Finger cap 210 is shaped so as to define a cavity for insertion of a human finger of a surgeon, and is typically shaped like a thimble. For example, the opening of the cavity may have a diameter of at least 1 cm, less than 2.5 cm, and/or between 1 and 2.5 cm, and the length of cavity along a central longitudinal axis 222 of finger cap 210 may be at least 1 cm, less than 4 cm, and/or between 1 and 4 cm. For some applications, finger cap 210 comprises a flexible material, such as silicone. Alternatively, finger cap 210 comprises a rigid material, such as plastic or metal.

For applications in which finger cap 210 comprises a rigid material, the finger cap may serve as stiff back support structure 13, and patch support 4 does not comprise a separate stiff back support structure 13. Alternatively, as mentioned above, the surgeon's finger, inserted into finger cap 210, serves to provide a stiff back support structure to yielding pad 15, and patch support 4 does not comprise a separate stiff back support structure 13. Typically, patch support 4 is fixed to finger cap 210. Alternatively, the patch support is provided separately to the surgeon, who couples the patch support to the finger cap.

Patch 3 is removably coupled to patch support 4 such that the patch rests against the yielding pad. The yielding pad enables finger applicator 200 to gently apply the patch to the blood vessel. For some applications, yielding pad 15 is shaped so as to define one or more slits 17, into which respective edges of patch 3 are inserted in order to removably couple the patch to the patch support. When the second substance of the patch is brought in contact with the first substance, the patch adheres to the blood vessel.

For some applications, patch support 4 extends laterally from finger cap 210. For example, a line 220 normal to the surface of patch support 4 may form an angle of between 60 and 120 degrees, e.g., 90 degrees, with central longitudinal axis 222 of finger cap 210.

Reference is made to FIG. 13C, which is a schematic illustration of packaging of finger applicator 200, in accordance with an application of the present invention. In this application, finger applicator 200 is packaged in protective covering 30, with patch 3 removably coupled to patch support 4. For some applications, a kit is provided that comprises one or more packaged finger applicators 200 and container 27, as described hereinabove with reference to FIG. 1F. Container 27 contains a first component of an adhesive and not a second component of the adhesive; for example, the first component may comprise a viscous pre-gel. Patch 3 comprises the second component of the adhesive and not the first component of the adhesive.

In an application of the present invention, patch 3 is provided by the manufacturer in several sizes, such as small, medium, large, and extra-large, optionally preloaded in the applicator. Alternatively, a single size patch is provided by the manufacturer (e.g., 5″×5″), and, during the medical procedure, is cut to size on-site for the particular patient, and is placed in the applicator.

For some applications, the applicators described herein are single-use or semi-disposable. Alternatively, the applicators are configured to be reusable. For example, they may comprise a metal, such as stainless steel.

For some applications, the ends of the patch comprise Velcro to couple the ends to one another.

For some applications, in addition to using one or more patches 3 for applying the second component of the adhesive (e.g., the curing agent), as described hereinabove, one or more additional patches are provided for applying the first component of the adhesive (e.g., the pre-gel) to the tubular structure, e.g., blood vessel and/or synthetic graft. Optionally, techniques and applicators described herein are used for applying these additional patches. For some applications, these additional patches are removed from the tubular structure before applying patches 3 to the tubular structure, while for other applications, these additional, patches are left on the tubular structure, and patches 3 are placed at least partially on these additional patches.

In an experiment conducted by a portion of the inventors, performance of a patch applicator similar to the applicator described hereinabove with reference to FIGS. 1C-D was assessed. The patch applicator was used to apply patches to seal vascular-graft anastomotic suture lines on large peripheral blood vessels, such as carotid and femoral arteries, and jugular veins, in several pigs. The patch applicator comprised a forceps (comprising stainless steel) and two patch supports, which comprised respective yielding pads comprising rubber. The applicator was evaluated for in vivo sealing of vascular suture lines by application of a dual-component adhesive. A first component of the adhesive comprised a pre-gel, and a second component of the adhesive comprised a hardening mesh containing a curing agent.

Two patches, comprising respective sheets of the hardening mesh (containing the curing agent), were pre-attached to the yielding pads of the two patch supports (one patch per patch support). The applicator was positioned on the anastomotic site to which an adhesive pre-gel had been applied. The pre-attached patches were brought together to press on the pre-gel. The applicator was left closed for about one minute to apply mild pressure to the pre-gel treated site. The curing agent of the patches cured the pre-gel, causing the patches to adhere to the site. When the applicator was removed from the site, the patches were naturally released from the yielding pads of the patch supports and left incorporated into the pre-gel on the site. The applicator was retrieved from the body, while the adhesive (pre-gel and hardening mesh) completely cured.

Following the application of the adhesive, the time to hemostasis (TTH) was evaluated. The mean TTH was 0.25±0.4 minutes, without compromising the vascular supply of the treated area and with no other adverse events.

The scope of the present invention includes embodiments described in the following applications, which are assigned to the assignee of the present application and are incorporated herein by reference. For some applications, techniques and apparatus described in one or more of the following applications are combined with techniques and apparatus described herein:
International Application PCT/IL2008/001451, filed Nov. 5, 2008, entitled, “Adhering bandage and methods of applying the same,” which published as PCT Publication WO 09/060438;
International Application PCT/IL2008/001452, filed Nov. 5, 2008, entitled, “Adhesives and methods of applying the same,” which published as PCT Publication WO 09/060439; and
International Application PCT/IL2006/000289, filed Mar. 2, 2006, entitled, “Novel adhesive materials, manufacturing thereof, and applications thereof,” which published as PCT Publication WO 06/092798, and U.S. application Ser. No. 11/885,555, which published as US Patent Application Publication 2008/0167400, in the national stage thereof.

For some applications, the methods and applicators described herein are used, mutatis mutandis, to apply materials other than adhesive glues or curing agents.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a tubular structure in a body of a patient, the apparatus comprising:
an adhesive comprising first and second components;
a container, which contains the first component of the adhesive and not the second component of the adhesive;
one or more patches, which comprise the second component of the adhesive and not the first component of the adhesive; and
an applicator, which is configured to removably hold the one or more patches, and to place the one or more patches at least partially around the tubular structure.

2. (canceled)

3. The apparatus according to claim 1, wherein the applicator further comprises one or more patch supports, which are configured to removably hold the one or more patches.

4. (canceled)

5. The apparatus according to claim 1,
wherein the one or more patch supports comprise exactly two patch supports,
wherein the one or more patches comprise exactly two patches, which are removably coupled to respective surfaces of respective patch supports,
wherein the applicator comprises exactly two arms coupled to one another, and
wherein the arms and the patch supports are configured such that when the two patch supports are coupled to the two arms, respectively, the surfaces of the patch supports face each other.

6. (canceled)

7. The apparatus according to claim 3, wherein each of the patch supports comprises a stiff back support structure, and a yielding pad coupled to the stiff back support structure, and wherein one of the one or more patches is removably coupled to the patch support such that the patch rests against the yielding pad, and the yielding pad is disposed between the patch and the patch support.

8. (canceled)

9. The apparatus according to claim 1, wherein the applicator comprises one or more patch supports, which are configured to hold the one or more patches, and wherein the apparatus further comprises one or more protective coverings, each of which packages one of the one or more patch supports and one of the one or more patches, and not the applicator.

10-16. (canceled)

17. The apparatus according to claim 1, wherein the second component of the adhesive comprises a curing agent.

18. The apparatus according to claim 17, wherein the first component of the adhesive comprises a pre-gel.

19-21. (canceled)

22. A method comprising:
applying a first component of an adhesive to a tubular structure in a body of a patient; and
placing one or more patches, which comprise a second component of the adhesive, at least partially around the tubular structure, using an applicator that initially holds at least a portion of the one or more patches, such that the second component of the adhesive comes in contact with the first component of the adhesive.

23. (canceled)

24. The method according to claim 22, wherein the applicator includes one or more patch supports, and wherein placing comprises placing the one or more patches while the one or more patches are removably coupled to the one or more patches supports.

25-34. (canceled)

35. The method according to claim 22, wherein the second component of the adhesive comprises a curing agent, and wherein placing the one or more patches comprises bringing the curing agent into contact with the first component of the adhesive.

36. The method according to claim 35, wherein the first component of the adhesive comprises a pre-gel, and wherein applying the first component comprises applying the pre-gel to the tubular structure.

37. (canceled)

38. The method according to claim 22, wherein the tubular structure is one or more structures selected from the group consisting of: at least one blood vessel, and at least one synthetic graft.

39-40. (canceled)

41. Apparatus comprising:
a patch, which comprises a component of an adhesive; and
a finger applicator, which comprises a finger-coupling element, and a patch support to which the patch is removably coupled.

42-45. (canceled)

46. The apparatus according to claim 41, wherein the patch support comprises a yielding pad, to which the patch is removably coupled.

47. The apparatus according to claim 46, wherein the patch support further comprises a stiff back support structure.

48-51. (canceled)

52. A method comprising:
coupling a finger applicator to a human finger using a finger-coupling element of the finger applicator; and
placing a patch, which comprises a component of an adhesive, at least partially around a tubular structure in a body of a patient, using the finger applicator while the patch is removably coupled to a patch support of the finger applicator.

53-55. (canceled)

56. The method according to claim 52, wherein the patch support includes a yielding pad, to which the patch is removably coupled.

57. The method according to claim 56, wherein the patch support further includes a stiff back support structure.

58-69. (canceled)

70. The apparatus according to claim 1, wherein the second component of the adhesive is absorbed into the one or more patches.

71. The apparatus according to claim 1, wherein the second component of the adhesive comprises a solid.

72. The apparatus according to claim 1, wherein the one or more patches comprise a material selected from the group consisting of: a knitted mesh of fabric, and gauze.

73. The apparatus according to claim 1, wherein the one or more patches are biodegradable.

74. The apparatus according to claim 1, wherein the applicator is configured to fasten the one or more patches to the tubular structure.

75. The method according to claim 22, wherein placing comprises placing the one or more patches while the second component of the adhesive is absorbed into the one or more patches.

76. The method according to claim 22, wherein the second component of the adhesive comprises a solid, and wherein placing comprises placing the one or more patches that comprise the second solid component.

77. The method according to claim 22, wherein placing the one or more patches comprises removing the applicator while leaving the one or more patches at least partially around the tubular structure.

78. The method according to claim 77, wherein leaving comprises leaving the one or more patches at least partially around the tubular structure until the one or more patches biodegrade.

79. The method according to claim 22, wherein the one or more patches comprise a material selected from the group consisting of: a knitted mesh of fabric, and gauze, and wherein placing comprises placing the one or more patches that comprise the selected material.

80. The method according to claim 22, wherein placing the one or more patches comprises fastening the one or more patches to the tubular structure.

81. The apparatus according to claim 41, wherein the component of the adhesive is absorbed into the patch.

82. The apparatus according to claim 41, wherein the component of the adhesive comprises a solid.

83. The apparatus according to claim 41, wherein the patch comprises a material selected from the group consisting of: a knitted mesh of fabric, and gauze.

84. The apparatus according to claim 41, wherein the patch is biodegradable.

85. The apparatus according to claim 41, wherein the finger applicator is configured to fasten the patch to a tubular structure.

86. The method according to claim 52, wherein placing comprises placing the patch while the component of the adhesive is absorbed into the patch.

87. The method according to claim 52, wherein the component of the adhesive comprises a solid, and wherein placing comprises placing the patch that comprises the second solid component.

88. The method according to claim 52, wherein placing the patch comprises removing the applicator while leaving the patch at least partially around the tubular structure.

89. The method according to claim 88, wherein leaving comprises leaving the patch at least partially around the tubular structure until the patch biodegrades.

90. The method according to claim 52, wherein the patch comprises a material selected from the group consisting of: a knitted mesh of fabric, and gauze, and wherein placing comprises placing the patch that comprises the selected material.

91. The method according to claim 52, wherein placing the patch comprises fastening the patch to the tubular structure.
